(19) United States
(12) Patent Application Publication
(10) Pub. No.: US 2026/0250716 A1
(43) Pub. Date: Aug. 27, 2026

(54) METHODS OF ELICITING IMMUNO-TOLERANCE USING SOLUBLE IMMUNE CHECKPOINT PROTEINS

(71) Applicant: Duke University, Durham, NC (US)

(72) Inventors: Zachary HARTMAN, Durham, NC (US); Dawn BOWLES, Durham, NC (US); Herbert LYERLY, Durham, NC (US); Carmelo MILANO, Durham, NC (US)

(21) Appl. No.: 19/117,304

(22) PCT Filed: Sep. 29, 2023
(86) PCT No.: PCT/US2023/075614

§ 371 Date: Mar. 31, 2025

Related U.S. Application Data
(60) Provisional application No. 63/411,298, filed on Sep. 29, 2022.

(51) Int. Cl. C12N 15/864; A61K 31/711; C07K 14/705

(52) U.S. Cl. CPC C12N 15/8645; A61K 31/711; C07K 14/70521; C07K 14/70532; C07K 2319/036; C12N 2750/14132; C12N 2750/14143; C12N 2830/002; C12N 2830/008; C12N 2830/34

(57) ABSTRACT

The present disclosure provides, in part, methods of eliciting immuno-tolerance using truncated membrane-bound and soluble immune checkpoints. Constructs comprising the immune checkpoint protein or portions thereof capable of binding to and activating the immune checkpoint proteins receptor are provided. The nucleic acid encoding the immune checkpoint protein or portion thereof is operably connected to a promoter and optionally also a secretory signal to allow for secretion of the immune checkpoint protein or portion thereof. The constructs may include a viral vector which can be used to deliver or introduce the construct into a transplantable article or organ.

### CROSS-REFERENCE TO RELATED APPLICATIONS

This application claims priority to U.S. Provisional Application No. 63/411,298 filed on Sep. 29, 2022, the contents of which are incorporated by reference in their entireties.

### REFERENCE TO AN ELECTRONIC SEQUENCE LISTING

The contents of the electronic sequence listing (155554.00711.xml; Size: 18,509 bytes; and Date of Creation: Sep. 29, 2023) is herein incorporated by reference in its entirety.

### BACKGROUND

The number of patients with heart failure is expected to increase by nearly 46% by 2030 to approximately 8 million. Nearly 50% of these patients are expected to die within 5 years of diagnosis. For those with end stage heart failure, heart transplantation is the gold standard treatment. Long-term survival following transplant is limited due to graft dysfunction, rejection, vasculopathy, and chronic systemic immunosuppression. There is a strong unmet need to apply major advances in gene therapy to the field of transplantation to decrease transplant rejection, decrease use of immunosuppressants, and expand the donor pool. In view of the foregoing, it would be desirable to provide new targets and methods to minimize organ rejection.

### SUMMARY

The present disclosure provides constructs encoding immune checkpoint proteins, including PDL1 and methods of using the constructs to transduce organs and reduce transplant rejection.

One aspect of the present disclosure provides a nucleic acid construct comprising a promotor operably connected to a nucleic acid sequence encoding an immune checkpoint protein or portion thereof including the extracellular portion of an immune checkpoint protein, wherein the immune checkpoint protein or portion thereof including the extracellular portion of an immune checkpoint protein is capable of binding to its receptor. In some embodiments, the construct comprises a secretory signal operably connected to the promoter and functionally linked to the nucleic acid encoding the immune checkpoint protein or portion thereof. In some embodiments, the immune checkpoint protein comprises PDL1 or PD1. In some embodiments, the construct comprises a viral vector, for example an AAV vector. In some embodiments, the composition comprises a pharmaceutical composition comprising the construct described herein.

A second aspect of the present disclosure provides a method for expressing an immune checkpoint protein or portion thereof in a transplantable article. In some embodiments, the method comprises introducing the construct or the pharmaceutical composition described herein into a transplantable article. In some embodiments, the method comprises ex vivo or in vitro perfusion of the construct or the pharmaceutical composition into the article. In some embodiments, the method further comprises transplanting the article into a subject. In some embodiments, the article is selected from the group consisting of an organ, a cell population, skin, and a tissue.

Another aspect of the present disclosure provides a method for preventing or reducing rejection of a transplanted article in a subject. In some embodiments, the method comprises introducing the construct or a pharmaceutical composition described herein into a transplantable article prior to transplantation of the article into the subject. In some embodiments, the method further comprises transplanting the article into a subject.

Another aspect of the present disclosure provides a method for introducing a construct into a transplantable article, the method comprising ex vivo perfusion of the construct into the article prior to transplantation.

Another aspect of the present disclosure provides a method for preventing or reducing rejection of a transplanted article in a subject. In some embodiments, the method comprises introducing a construct or a pharmaceutical composition described herein into a transplantable article prior to transplantation into a subject. In some embodiments, the method further comprises transplanting the article into a subject.

### BRIEF DESCRIPTION OF THE DRAWINGS

The present technology can be better understood by reference to the following drawings. The drawings are merely exemplary to illustrate certain features that may be used singularly or in combination with other features and the present technology should not be limited to the embodiments shown.

FIG. 1. Depiction of PD-L1 variant mediated T-cell deactivation. PD-L1 variant constructs include PD-L1 full length, PD-L1 ICD truncated, and soluble PD-L1.

FIG. 2. In vitro expression of PD-L1 full length, truncated, and secreted isoforms in triple negative breast cancer cells.

FIG. 3. Elevated PD-L1 (pg/mL) in serum of PD-L1 secreting isoform tumor-bearing mice.

FIG. 4. PD-L1 isoform expression enhances engraftment of immunogenic cell lines in mice.

FIG. 5. Depiction of a construct targeted to the ROSA 26 locus.

FIG. 6. In vivo PD-L1 expression is evident in transgenic mice with both the truncated and secreted variant confirmed by A) histology and B) ELISA.

FIG. 7. Transgenic mice containing the truncated PD-L1 construct demonstrate increased tolerance against rejection and prolonged graft survival.

FIG. 8. A viral vector with the PDL1-secreted construct introduced into the allograft prior to transplantation results in prolonged graft survival for greater than 80 days.

### DETAILED DESCRIPTION

The present disclosure is based, in part, on the discovery by the inventors of utilizing gene therapy approaches to transduce transplanted organs ex vivo or in vitro with immunosuppressive genes. Disclosed herein are constructs encoding immune checkpoint proteins, including PDL1 and methods of using the constructs to transduce organs and reduce transplant rejection.

### Constructs:

In a first aspect, the present invention provides a construct comprising a promotor operably connected to a nucleic acid sequence encoding an immune checkpoint protein or extracellular portion of an immune checkpoint protein. The immune checkpoint protein or extracellular portion of an immune checkpoint protein is capable of binding to its receptor and suitably activates the receptor to allow the immune checkpoint signaling to occur. A construct, or expression vector, may also be known as an expression construct, is usually a plasmid or virus designed for gene expression in cells. The construct is used to introduce a specific gene into a target cell and can commandeer the cell's mechanism for protein synthesis to produce the protein encoded by the gene.

The term “construct” or “polynucleotide construct” is a polynucleotide which allows the encoded sequence to be replicated and/or expressed in the target cell. A construct may contain an exogenous promoter, operably linked to any one of the polynucleotides described herein. As used herein, a polynucleotide is “operably connected” or “operably linked” when it is placed into a functional relationship with a second polynucleotide sequence. As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the construct is an expression construct, a vector or a viral vector. A vector is any particle used as a vehicle to artificially carry a foreign nucleic sequence, typically DNA into another cell, where it can be replicated and/or expressed. A construct containing foreign DNA is termed recombinant DNA. The four major types of constructs and vectors are plasmids, viral vectors, cosmids, and artificial chromosomes. Expression constructs comprise a heterologous promoter and the nucleic acid sequence encoding a protein of interest (e.g., immune checkpoint protein) which is capable of expression in the cell in which it is introduced. The expression constructs include constructs which are capable of directing the expression of exogenous genes to which they are operatively linked. Such constructs are referred to herein as “recombinant constructs,” “expression constructs,” “recombinant expression vectors” (or simply, “expression vectors” or “vectors”) and may be used interchangeably. Suitable constructs are known in the art and contain the necessary elements for the gene encoded within the construct to be expressed as a protein in the host cell. The term “vector” and “construct” are used interchangeably herein and refer to a nucleic acid molecule capable of transporting another nucleic acid to which it has been linked. One type of vector is a “plasmid”, which refers to a circular double stranded DNA loop into which additional DNA segments may be ligated, such as exogenous DNA segments encoding the mutant α-gal protein. Another type of vector is a viral vector, wherein additional DNA segments may be ligated into the viral genome. Viral vectors are incorporated into viral particles that are then used to transport the viral polynucleotide encoding the protein of interest into the target cells. Certain constructs are capable of autonomous replication in a host cell into which they are introduced. Other constructs can be integrated into the genome of a host cell upon introduction into the host cell, and thereby are replicated along with the host genome (e.g., lentiviral vectors). Moreover, certain vectors are capable of directing the expression of exogenous genes to which they are operatively linked. In general, constructs of utility in recombinant DNA techniques are often in the form of plasmids. In the present specification “vector” include expression vectors, such as viral vectors (e.g., replication defective retroviruses (including lentiviruses), adenoviruses and adeno-associated viruses (AAV)), which serve equivalent functions.

The constructs are heterogeneous exogenous constructs containing sequences from two or more different sources. Suitable constructs or vectors include, but are not limited to, plasmids, retroviruses, adenoviruses, oncoretroviruses, lentiviruses, spumaviruses, adeno-associated viruses, herpes simplex viruses, among others and includes constructs that are able to express the protein of interest. A preferred vector is an adeno-associated vector (AAV). Suitable methods of making viral particles are known in the art to be able to transform cells in order to express the protein of interest as described herein.

Heterologous promoters useful in the practice of the present invention include, but are not limited to, constitutive, inducible, temporally-regulated, developmentally regulated, chemically regulated, tissue-preferred, tissue-specific promoters and cell-type specific. The heterologous promoter may be an animal, bacterial, fungal, viral or synthetic promoter. Suitable promoters are known and described in the art. In mammalian cells, typical promoters include, without limitation, promoters for Rous sarcoma virus (RSV), human immunodeficiency virus (HIV-1), cytomegalovirus (CMV), SV40 virus, chicken beta actin, 3-phosphoglycerate kinase promoter, as well as the translational elongation factor EF-1α promoter or ubiquitin promoter.

In some embodiments, the constructs described herein may comprise a secretory signal. A secretory signal, sometimes called a signal peptide, signal sequence, targeting signal, localization signal, localization sequence, transit peptide, leader sequence or leader peptide, is or encodes a short peptide (usually 16-30 amino acids long) present at the N-terminus, C-terminus or internally of most newly synthesized proteins that are destined toward the secretory pathway. Often, a secretory signal functions to prompt a cell to translocate the protein to the cellular membrane and/or extracellular space. Signal peptides adhere to a generic three-domain structure, comprising a basic N-domain, a hydrophobic H-domain, and a slightly polar C-domain. As a representative example, the signal sequence from mouse follicular stimulating hormone B (5′-atgatgaagttgatccagctttgcatcttattctggtgctggagagcaatctgctgc-3′ SEQ ID NO: 13) was used in mouse constructs and experiments.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

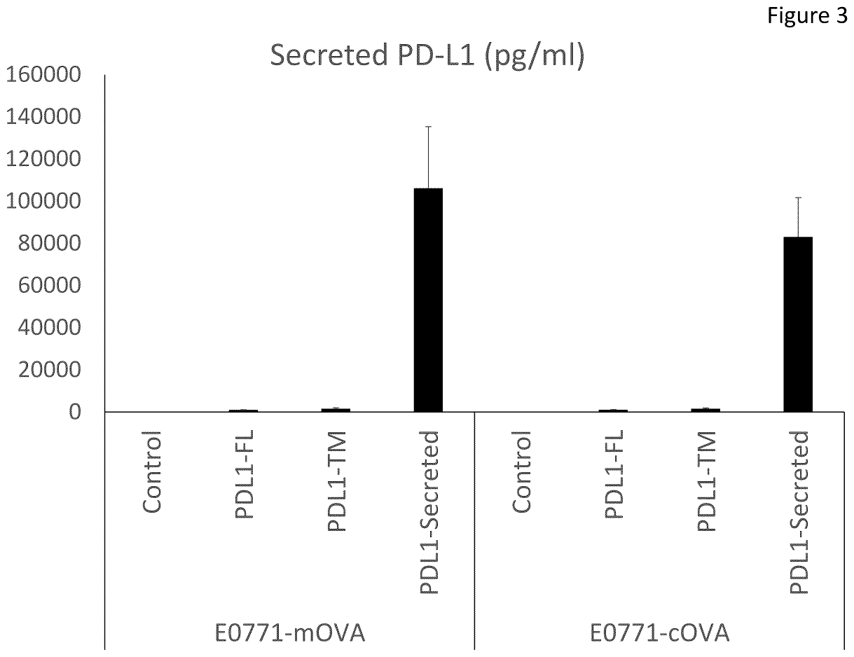

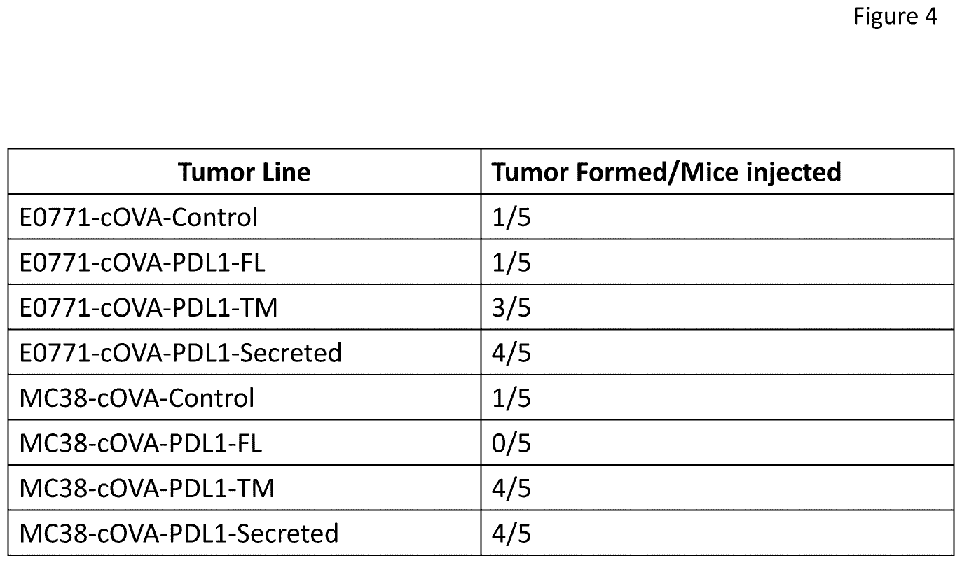

In some embodiments, the constructs described herein encode an immune checkpoint protein or extracellular portion of an immune checkpoint protein. Immune checkpoints are inhibitory regulators of the immune system that are crucial to maintaining self-tolerance, preventing autoimmunity, and controlling the duration and extent of immune responses in order to minimize collateral tissue damage. When the checkpoint protein and ligand partner protein, which are most often expressed on T cells, bind together, they send an “off” signal to the T cells, thus reducing the immune response. Immune checkpoint proteins are often overexpressed on tumor cells or on non-transformed cells within the tumor microenvironment and compromise the ability of the immune system to mount an effective anti-tumor response, thus allowing tumor cells to proliferate. Accordingly, immune checkpoint inhibitors are a standard of care for the treatment of some cancers. As shown in the Examples, overexpression or induced expression of the immune checkpoint proteins on non-HLA matched tumors allowed the tumor cells to multiply in a mouse model where the growth of the non-HLA typed tumor should have been inhibited by the immune response of the animal. See FIGS. 3 and 4. The immune checkpoint proteins lacking the transmembrane domain or prepared as a secreted form were significantly better at allowing tumor formation in a mouse model, suggesting these forms may be better suited to transfer into a transplantable article or organ to reduce the risk of immune-based rejection of the transplanted article or organ. Immune checkpoint proteins and their ligands include, without limitation, A2AR, A2BR, B7-H2, B7-H3, B7-H4, 2B4 (CD244), B7.1, B7.2, BTLA, CTLA4, ICOS, IDO, ITL-4, HVEM, KIR, LAG3, gp49B, NOX2, PD1, PDL1, PDL2, PIR-B, TIM-1, TIM-3, TIM-4, TIGIT, VISTA, SIGLEC7, CD47, CD48, CD39, CD73, CD160, CD200, HVEC, CEACAM1, CD155, LAG-3, HLA-E. In some embodiments, the immune checkpoint protein is CTLA4.

In some embodiments, an immune checkpoint protein or extracellular portion of an immune checkpoint protein described herein is capable of binding to its receptor. Ligand receptor interactions, or binding results in a molecular response. The binding of an immune checkpoint protein to its receptor may result in a change in immune function, cytokine production, proliferation, cellular migration or signaling. Some immune checkpoint proteins are able to interact with more than one receptor, or more than one ligand. For example, CTLA4 is a receptor for CD80 as well as CD86. The truncated (portions of) immune checkpoint proteins provided herein may remain able to cause the same or significantly the same change in immune function as the full-length protein.

In some embodiments, the construct comprises the immune checkpoint protein programmed death-ligand 1 (PD-L1) also known as cluster of differentiation 274 (CD274) or B7 homolog 1 (B7-H1) or programmed cell death protein 1, also known as PD-1 and CD279. Engagement of PD-L1 with its receptor PD-1 on T cells delivers a signal that inhibits TCR-mediated activation of IL-2 production and T cell proliferation, this down-regulates the immune system and promoting self-tolerance by suppressing T cell inflammatory activity. PD-L1 is a type I transmembrane glycoprotein encoded by the CD274 gene on chromosome 9 in humans. Transcription of this gene can produce multiple PD-L1 splice variants, including PD-L1 lncRNA splice isoforms, truncated PD-L1 and soluble PD-L1. In some embodiments, the constructs described herein may comprise full length PD-L1, including SEQ ID NO: 1 (mouse, DNA) or SEQ ID NO: 2 (mouse, amino acid), SEQ ID NO: 7 (human, DNA) or SEQ ID NO: 8 (human, amino acid); PD-L1 truncated after the transmembrane domain, including SEQ ID NO: 3 (mouse, DNA) or SEQ ID NO: 4 (mouse, amino acid), SEQ ID NO: 9 (human, DNA) or SEQ ID NO: 10 (human, amino acid); and/or secreted PD-L1, including SEQ ID NO: 5 (mouse, DNA) or SEQ ID NO: 5 (mouse, amino acid), SEQ ID NO: 11 (human, DNA) or SEQ ID NO: 12 (human, amino acid).

In some embodiments of the present disclosure, a pharmaceutical composition comprising a construct described herein and a pharmaceutically acceptable carrier, diluent and/or excipient. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

In another embodiment, the present formulation may also comprise other suitable agents such as a stabilizing delivery vehicle, carrier, support or complex-forming species. The coordinate administration methods and combinatorial formulations of the instant invention may optionally incorporate effective carriers, processing agents, or delivery vehicles, to provide improved formulations for delivery of the constructs or vectors described herein.

The pharmaceutical formulation may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final pharmaceutical formulation.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservative.

### Methods

In a second aspect, the present invention provides methods for introducing one or more of the constructs described herein into a transplantable article. In some embodiments, the methods comprise introducing any of the constructs described herein or a pharmaceutical composition described herein into a transplantable article, optionally wherein introducing includes ex vivo or in vitro perfusion of the construct or the pharmaceutical composition into the article.

The constructs described herein may be introduced into an article for purposes of expressing the immune checkpoint protein of the construct. The construct can be introduced into the cell by any means known in the art. These means may include transfection or transduction. Transfection is the process of introducing nucleic acids into cells by non-viral methods. Transduction is the process whereby foreign DNA is introduced into another cell via a viral vector. These are common tools to introduce a foreign gene into host cells. Among others, additional means include transformation, and conjugation. In addition, methods employing targeted endonucleases to knock-in the construct may also be used such as CRISPR/Cas gene editing.

In some embodiments, the construct may be introduced to the article by ex vivo perfusion. Ex vivo perfusion is also called normothermic perfusion and comprises a machine which keeps organs at body temperature by continuously pumping or perfusing blood or a bloodless solution of nutrients, proteins and oxygen, through them. Ex vivo perfusion may reduce ischemic injury time and allow for graft evaluation. In some embodiments, the construct described herein may be included in the solution being perfused through the article.

In some embodiments, the method further comprises analyzing the article or cells of the article for the presence or expression of the construct or the immune checkpoint protein encoded by the construct in the article. Assessment of the construct or immune checkpoint protein may be by any means known in the art. Results of the assessment may be used to inform dosage, function or effect of the construct or may be useful in determining the levels or requirements for additional immunosuppressive treatments if the article is used as a transplant organ.

In some embodiments, an article may consist of an organ, a cell population, and a tissue. The article for transplantation may comprise an organ. The organ may include, without limitation heart, heart valves, lung, kidney, liver, pancreas, skin, spleen, middle ear, connective tissue, intestine, colon, eye, stomach, ovary, testes, bladder, uterus and adrenal glands. The cell population may comprise a stem cells, bone marrow and immune cells. Tissues may comprise bones tendons, ligaments, skin, heart valves blood vessels, pancreas islets, nerves, veins, and limbs.

In some embodiments, the method further comprises transplanting the article into a subject. Organ transplantation is a medical procedure in which an organ is removed from one body and placed in the body of a recipient, to replace a damaged or missing organ. The donor and recipient may be at the same location, or organs may be transported from a donor site to another location. An allograft is a transplant of an organ or tissue between two genetically non-identical members of the same species. Due to the genetic difference between the organ and the recipient, the recipient's immune system may identify the organ as foreign and attempt to destroy it, causing transplant rejection. In addition, in cases of stem cell, bone marrow or other hematopoietic transplants the immune cells of the transplant attack the host cells. This is called Graft-versus-host disease (GvHD). “Graft” refers to transplanted, or donated tissue, and “host” refers to the tissues of the recipient. Transplantation recipients often receive prophylactic treatment to suppress the immune system after the transplant. These treatments continue after transplantation. Immunosuppressant treatment includes, without limitation, Ruxolitinib, Belumosudil, Ibrutinib, corticosteroids as well as photopheresis. Human leukocyte antigen (HLA) typing or HLA matching is used to match recipients and donors for transplants. HLA are proteins found on most cells in your body and are used by the immune system to recognize foreign cells. HLA genes of the donor and recipient must be the same or match as closely as possible for transplantation to be successful and to lessen the chance of developing GvHD or transplant rejection. Two main classes of HLA antigens are recognized: HLA class I and HLA class II. HLA class I antigens (A, B, and C in humans) render each cell recognizable as “self,” whereas HLA class II antigens (DR, DP, and DQ in humans) stimulate the immune system. The methods provided herein may allow for an increased level of mismatch in HLA types between donors and recipients to allow for broader use of donated organs or may reduce the need for or the levels of immunosuppressant treatment needed to avoid transplant rejection of GvHD.

Another aspect of the present invention provides an ex vivo method for prevention or reducing rejection of a transplanted article in a subject. In some embodiment, the methods comprise introducing a construct into a transplantable article prior to transplantation into a subject. The constructs encode a promotor operably connected to a nucleic acid sequence encoding an immune checkpoint protein or extracellular portion of an immune checkpoint protein. The immune checkpoint protein or extracellular portion of an immune checkpoint protein is capable of binding to its receptor and may activate the receptor similarly to the native immune checkpoint protein.

A “subject in need thereof” as utilized herein may refer to a subject in need of treatment for a disease or disorder associated with organ transplantation. A subject in need thereof may include a subject having any condition in which a organ, cell or tissue transplant would be beneficial. The subject may be experiencing failure of or damage to the heart, lung, kidney, liver, pancreas, spleen, intestine, colon, eye, stomach, ovary, testes, bladder, uterus, adrenal glands, skin or any other organ or cell type in the body. The term “subject” may be used interchangeably with the terms “individual” and “patient” and includes human and non-human mammalian subjects.

In some embodiments, the compositions and methods provided herein may be utilized in xenotransplantation. Xenotransplantation, or heterologous transplant, is the transplantation, implantation or infusion of living cells, tissues or organs from one species to another, for example from a nonhuman animal source to a human recipient. Such cells, tissues or organs are called xenografts or xenotransplants. Nonhuman organs, cells or tissue may be genetically modified through ex vivo viral, or non-viral transduction with vectors permitting the expression of PD-11 genes and variants as described herein. Such approach may suppress the immune mechanism response for organ rejection and allow for more successful xenotransplantation.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

There is strong unmet need to apply major advances in gene therapy to the field of transplantation to decrease transplant rejection, decrease use of immunosuppressants, and expand the donor pool. The development of successful gene therapy strategies relies upon four components: 1) a delivery approach, 2) a vector (usually viral based), 3) a well-defined disease or injury state and disease models, and 4) molecular targets appropriately therapeutic for the disease or indication. Previously, the lack of an efficient delivery approach for targeted delivery of transgenes to donor hearts was a barrier to gene therapy for cardiac transplantation. Recently an ex vivo warm blood perfusion system (TransMedics, Organ Care System (OCS)) has been FDA-approved to prolong donor heart transport times and decrease ischemic injury. This system can also be opportunistically used for viral vector delivery to genetically modify transplanted organs prior to implantation. The inventors have successfully demonstrated the ability to deliver reporter genes by viral vectors homogenously to the entire myocardium of allografts used in porcine transplantation models.² Ex vivo warm blood perfusion mediated viral vector delivery to the heart is well-established in the inventor's laboratory.

In the following example, the inventors describe therapeutic targets to use in a gene therapy to minimize organ rejection. The role of T cells in the setting of transplant rejection has been well established as have a number of co-stimulatory pathways that mediate T cell activation. Organ rejection is primarily driven by T cells which recognize of foreign alloantigens on the donor organ. This recognition leads to subsequent effector responses and eventually organ damage and rejection. There are, however, a number of suppressive pathways to limit T cell activity. Among these, the PD-1/PD-L1 axis is a central pathway for CD8+ T cell suppression. Therapies targeting the PD-1/PD-L1 pathways through a series of blocking antibodies have revolutionized clinical practice for cancer treatment. Interestingly, it has been reported that in cancer patients with cardiac transplants, the use of PD-1/PD-L1 blocking antibodies is associated with both cardiac and kidney allograft rejection.^{3,4} In a recent investigation of biopsies from acutely and chronically rejected human heart transplants, the inventors observed dysregulated protein expression of PD-1 and PD-L1, supporting the relevance of this pathway in cardiac rejection.⁵ Bracamonte-Baran and colleagues demonstrated that allograft endothelial PD-L1 expression was inversely correlated with CD8+ T cell infiltration in 23 endomyocardial biopsies.⁶ In addition, PD-L1 transgene expression in a transgenic pig model resulted in reduced capacity to stimulate proliferation of CD4+ T cells.⁷ Pre-clinical experiments by others using donor PD-L1 knockout and recipient PD-1 knockout mice have determined that cardiac allograft rejection was significantly accelerated by the lack of PD-L1.⁸⁻¹⁰ Conversely, PD-1 overexpression in mouse T cells prior to heart transplantation demonstrated both a graft survival benefit and mitigation of rejection.¹¹ This survival benefit was dependent on PD-L1 expression on the donor allograft—transplants from PD-L1 knockout donor mice and PD-1 overexpressing T cells in recipient mice demonstrated no survival benefit. In a clinical setting, however, overexpression of organ-specific PD-L1 rather than systemic T cells would be of more utility to patients and minimize potential systemic adverse effects. The prevention of immune allograft destruction has been demonstrated in a transgenic pancreatic islet transplant murine model even in the absence of immunosuppression for greater than 50 weeks.¹² Whether cardiac PD-L1 overexpression could reverse heart allograft rejection remains unexplored.

### Results

Molecular targets for transplant rejection. In a recent investigation of cardiac specimens from 18 rejected and non-rejected human heart transplants at our institution, we found dysregulated expression of the PD-1/PD-L1 pathway supporting its relevance in cardiac rejection.⁵ Acute rejection was associated with near absent PD-L1 in myocytes. PD-L1 was also significantly lower in lymphocyte populations compared to PD-1. A trend of decreased PD-L1 in cardiac myocytes compared to PD-1 was observed. However, these studies were conducted in rejected allografts that had already been exposed to immunosuppression.

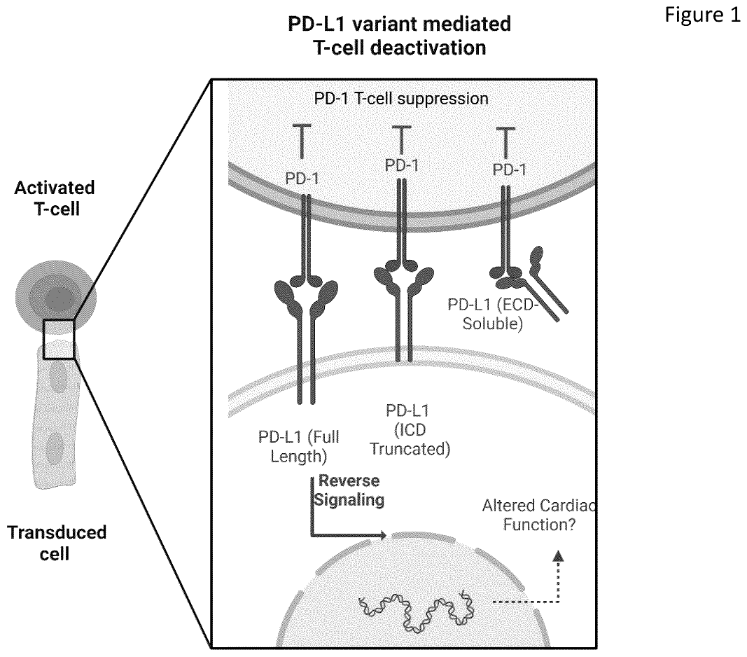

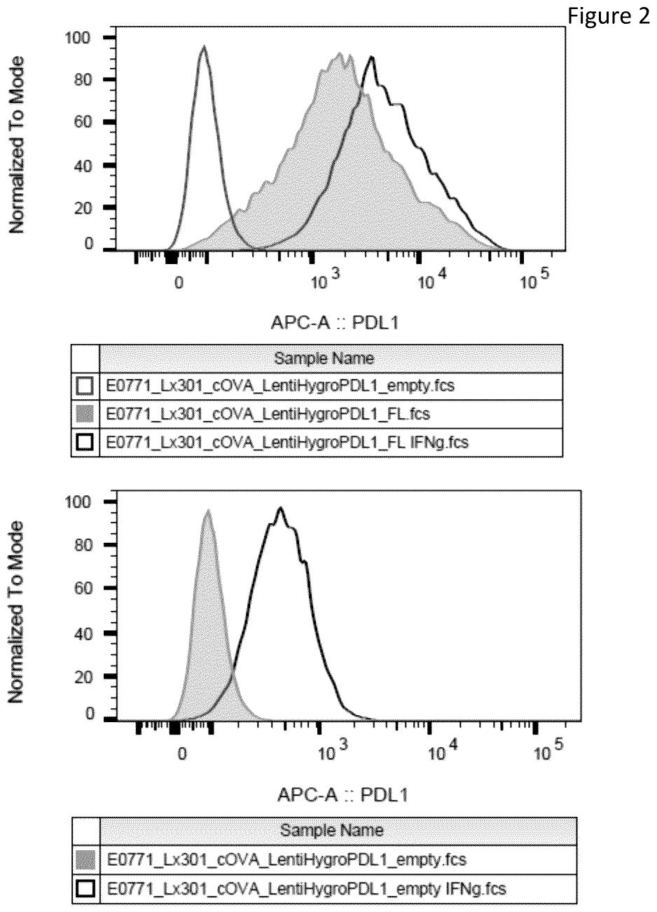

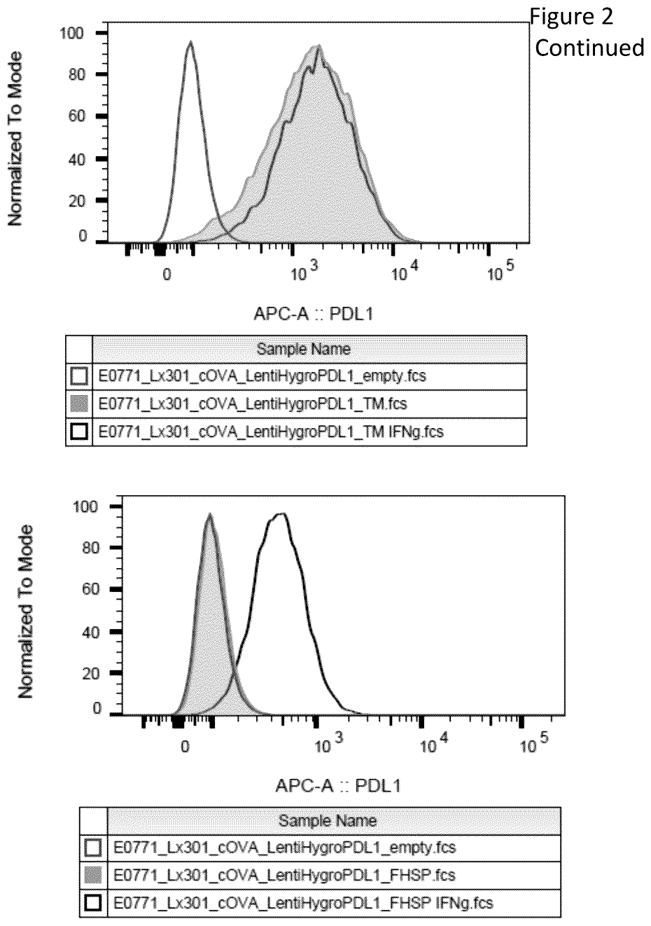

Cell Based models to assess ability of PD-L1 variant ability to resist cellular rejection. The PD-L1 protein has three main components: an intracellular domain, extracellular domain, and a transmembrane domain. The intracellular domain enables reverse signaling (upon binding to PD-1) that can alter a variety of cellular behaviors. However, these have not been well described.¹³ These could potentially include negative feedback loops that may limit the effectiveness of gene therapy aimed at overexpressing PD-L1. In addition, a secreted isoform of PD-L1 has also been described in human studies; this soluble form of PD-L1 has been reported to be elevated during pregnancy¹⁴ and has been implicated with poor outcomes in different cancers.¹⁵ Plasmid constructs containing the three PD-L1 variants-full length, truncated intracellular domain, and secreted isoform (FIG. 1). Lentiviral vectors encoding these construct under the control of the CMV promoter were generated and used to transduce different tumor models and have been confirmed to allow for robust expression in vitro (FIG. 2) and in vivo (FIG. 3). To assess the ability of these genes to resist immune mediated T cell attack, they were engineered into mammary cancer (E0771) or colorectal cancer (MC38) cells that also expressed an immunogenic form of ovalbumin (membrane associated) that could trigger rejection. These cells were then implanted to determine which genes could resist immune attack against syngeneic C57B16/J cells, evidenced by the ability of tumors to form in mice (FIG. 4). We found that control cells infected with an empty vector were rejected in 80% of cases, which was notably comparable to cells containing the full length version of PDL1 in both colorectal and mammary cells. Notably, tumor cells containing the truncated version of PDL1 and those containing the secreted version resisted rejection in the majority of mice, suggesting that a lack of reverse signaling were essential to establishing local immune suppression, perhaps mediated by the absence of particular feedback signaling loops.

### REFERENCES

1. Benjamin, E. J. et al. Heart Disease and Stroke Statistics-2017 Update: A Report From the American Heart Association. Circulation 135, e146-e603.
2. Bishawi et al. A normothermic ex vivo organ perfusion delivery method for cardiac transplantation gene therapy. Sci Rep. 2019 May 29; 9(1):8029. doi: 10.1038/s41598-019-43737-y.
3. Owonikoko, et al. Cardiac allograft rejection as a complication of PD-1 checkpoint blockade for cancer immunotherapy: a case report. Cancer Immunol Immunother. 2017 January; 66(1):45-50. doi: 10.1007/s00262-016-1918-2. Epub 2016 Oct. 22.
4. Lipson et al. Tumor Regression and Allograft Rejection after Administration of Anti-PD-1. N Engl J Med. 2016 Mar. 3; 374(9):896-8. doi: 10.1056/NEJMc1509268.
5. Bishawi et al. PD-1 and PD-L1 expression in cardiac transplantation. Cardiovasc Pathol. September-October 2021; 54:107331. doi: 10.1016/j.carpath.2021.107331. Epub 2021 Mar. 16.
6. Bracamonte-Baran, W, et al. Endothelial Stromal PD-L1 (Programmed Death Ligand 1) Modulates CD8+T-Cell Infiltration After Heart Transplantation. Circ Heart Fail. 2021 October; 14(10):e007982. doi: 10.1161/CIRCHEARTFAILURE.120.007982.
7. Buermann A, et al. Pigs expressing the human inhibitory ligand PD-L1 (CD 274) provide a new source of xenogeneic cells and tissues with low immunogenic properties. Xenotransplantation. 2018 September; 25(5):e12387. doi: 10.1111/xen.12387.
8. Ozkaynak et al. Programmed death-1 targeting can promote allograft survival. J Immunol. 2002 Dec. 1; 169(11):6546-53. doi: 10.4049/jimmunol.169.11.6546.
9. Yang et al. Critical role of donor tissue expression of programmed death ligand-1 in regulating cardiac allograft rejection and vasculopathy. Circulation. 2008 Feb. 5; 117(5):660-9. doi: 10.1161/CIRCULATIONAHA.107.741025. Epub 2008 Jan. 22.
10. Wang et al. Protective role of programmed death 1 ligand 1 (PD-L1) in nonobese diabetic mice: the paradox in transgenic models. Diabetes. 2008 July; 57(7):1861-9. doi: 10.2337/db07-1260. Epub 2008 Apr. 16.
11. Borges, T J, et al. Overexpression of PD-1 on T cells promotes tolerance in cardiac transplantation via ICOS-dependent mechanisms. JCI Insight. 2021 Dec. 22; 6(24):e142909. doi: 10.1172/jci.insight. 142909.
12. Paul P K, et al. Islet allografts expressing a PD-L1 and IDO fusion protein evade immune rejection and reverse preexisting diabetes in immunocompetent mice without systemic immunosuppression. Am J Transplant. 2022 Jul. 27. doi: 10.1111/ajt.17162.
13. Lucas et al. PD-L1 Reverse Signaling in Dermal Dendritic Cells Promotes Dendritic Cell Migration Required for Skin Immunity. Cell Rep. 2020 Oct. 13; 33(2):108258. doi: 10.1016/j.celrep.2020.108258.
14. Okuyama et al. Elevated Soluble PD-L1 in Pregnant Women's Serum Suppresses the Immune Reaction. Front Immunol. 2019 Feb. 18; 10:86. doi: 10.3389/fimmu.2019.00086. eCollection 2019.
15. Han et al. The clinical implication of soluble PD-L1 (sPD-L1) in patients with breast cancer and its biological function in regulating the function of T lymphocyte. Cancer Immunol Immunother. 2021 October; 70(10):2893-2909. doi: 10.1007/s00262-021-02898-4. Epub 2021 Mar. 10.

### Example 2

Targeted Allograft-Specific PD-L1 Overexpression Will Increase Time to Rejection after Heterotopic Cardiac Transplantation in a Transgenic Mouse Model

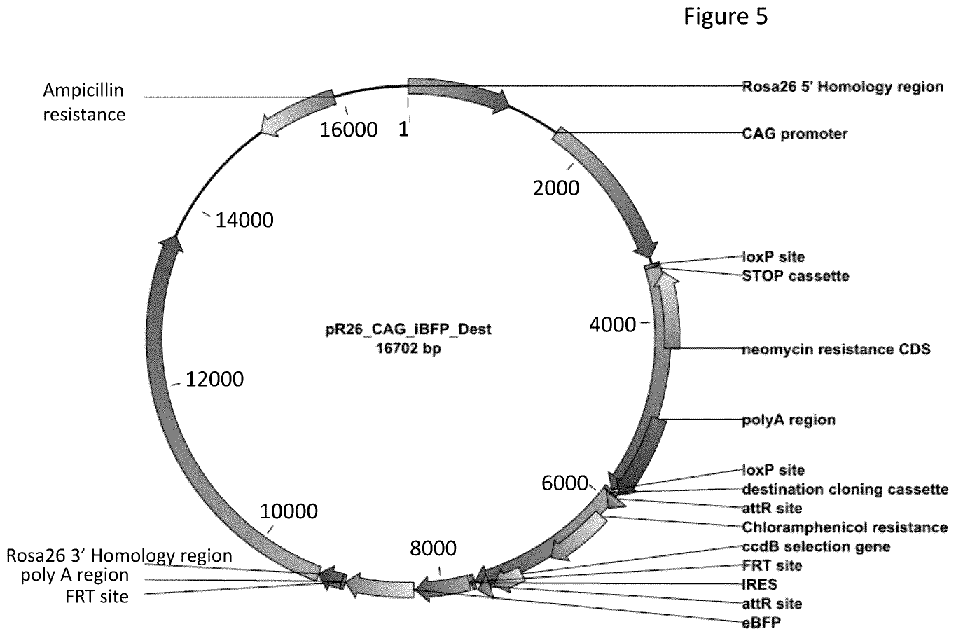

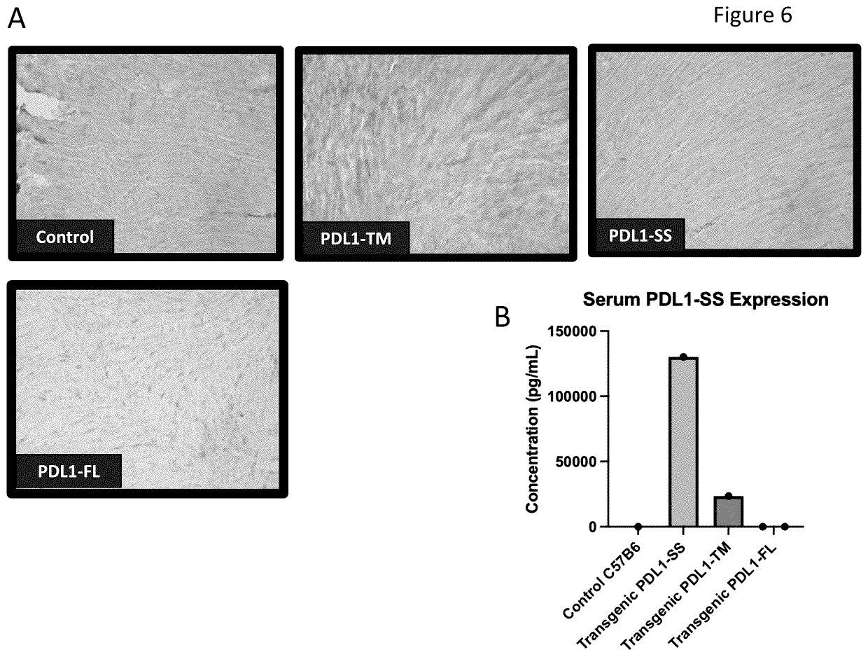

After demonstrating the utility of PD-L1 variants to suppress cellular immune responses in a superior manner to a full-length PD-L1, we sought to test their potential in mouse animal models through the generation of transgenic mice, as well as through vector-based transduction studies. To generate mice, we cloned PD-L1 (full length), PD-L1-TM (ICD truncated), and PD-L1-SS (soluble version) into a ROSA targeting vector, equipped with a CAG promoter that was activated after CRE recombination (FIG. 5). These plasmids were generated and sequenced verified before electroporation into embryonic stem cells and selection using G418. After selection, positive clones were screen through multi-plex PCR and targeted, intact clones verified. These ES cells were utilized to generate chimeric mice and these mice bred to established germline transmission of these transgenes. Upon transgenesis, mice were bred to Myh6CRE mice. After the generation of multiple litters, it was determined that PD-L1×Myh6-cre resulted in embryonic lethality, without any viable double positive mice, in contrast to mendelian ratios observed of PDL1-FL mice crossed to non-transgenic C57BL6/J mice. In contrast, we did observe that PD-L1-TM and PD-L1-SS mice could arise after crossing to Myh6-CRE, indicating a non-lethal and potentially more beneficial cardiac phenotype. To determine if this was related to CRE expression in the developing heart (or other tissues), we additionally crossed mice to Myh6-CRE/ER mice that would allow for temporal control of CRE after tamoxifen administration. In this setting, we were able to generate mendelian ratios of transgenic mice, indicating that a lack of expression resulted in embryonic lethality of PD-L1-FL. To examine the temporal control of PD-L1 expression, tamoxifen was administered for five days (75 mg/kg) and heart tissue assessed 1-week post-terminal tamoxifen injection (12 days after the first injection). These studies demonstrated that PD-L1-TM had robust PDL1 expression on cardiomyocytes by IHC, in comparison to PDL1-FL and PDL1-SS (FIG. 6). Using PD-L1 ELISA, we could also detect significant quantities of PD-L1 in the serum of mice with PD-L1-SS, some increase from PD-L1-TM, but no appreciable expression from PD-L1-FL or control non-transgenic mice. We thus conclude that secondary mechanisms to suppress PD-L1 expression by signaling from its intracellular domain (ICD) may be suppressing expression, as has been found to influence several different signaling pathways in cancer models (Jalali et al., Blood Cancer J 2019; Tamburini, Cell Reports 2020). As such, the demonstration of a lack of tissue-specific PD-L1 expression indicates a potential barrier to success for this strategy mediated by protein expression through ICD control that was unexpected but of essential importance in pursing this approach.

To determine if transgenic hearts could suppress immune rejection, we next initiated studies to perform major mismatch transplants between transgenic and non-transgenic hearts.

### Experimental Design

The donor Myh6-Cre/PD-L1 transgenic mice have a C57BL6 background and recipient mice are wild type BALB/c. There will be three experimental groups-one experimental group for donors of each transgenic PD-L1 variant and a HLA mismatch wild type positive control (C57BL6 donor heart into a BALB/c recipient). Heterotopic heart transplantation will be performed as follows: The donor mouse will be anesthetized and prepared. After sternotomy, the heart is explanted in sterile fashion. The heart will be arrested with cold buffer delivered into the aortic root. The heart will then be implanted in an HLA mismatched recipient. The ascending aorta of the donor will be attached in an end-to-side fashion to the abdominal aorta of the recipient, and the donor pulmonary artery will be attached in an end-to-side fashion to the recipient's inferior vena cava. The graft will be de-aired. The abdomen will be closed and the recipient mouse is recovered.

Assessment of Rejection: Assessment of cardiac rejection will primarily by three methods: 1) Daily physical exam. Graft function can be easily assessed with simple palpation on the animal's abdomen and graded on a 4-point standard International Society for Heart and Lung Transplantation scale¹⁶ 2) Echocardiography weekly 3) Subsets of animals (n=5 per time point) will be euthanized for blood and tissue weekly. Peripheral blood mononuclear cells (PBMCs) will be isolated and stored for immune profiling. Immune profiling will be performed via flow cytometry to characterize the cellular composition of immune responses during rejection when PD-L1 is overexpressed as compared to control animals. Hearts will be harvested and processed for sectioning and H&E staining. Mice will be continuously examined for intimal hyperplasia or other signs of cardiac allograft occlusive vasculopathy/rejection.

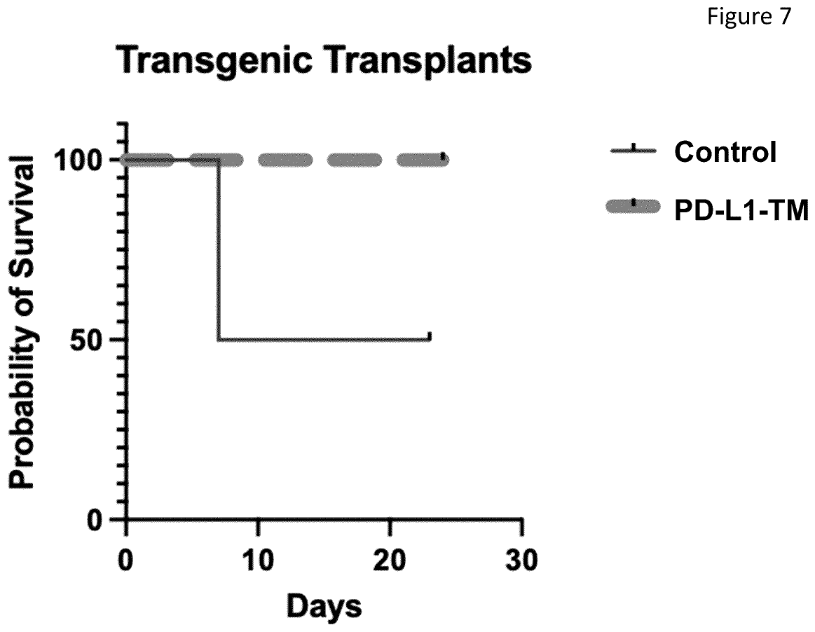

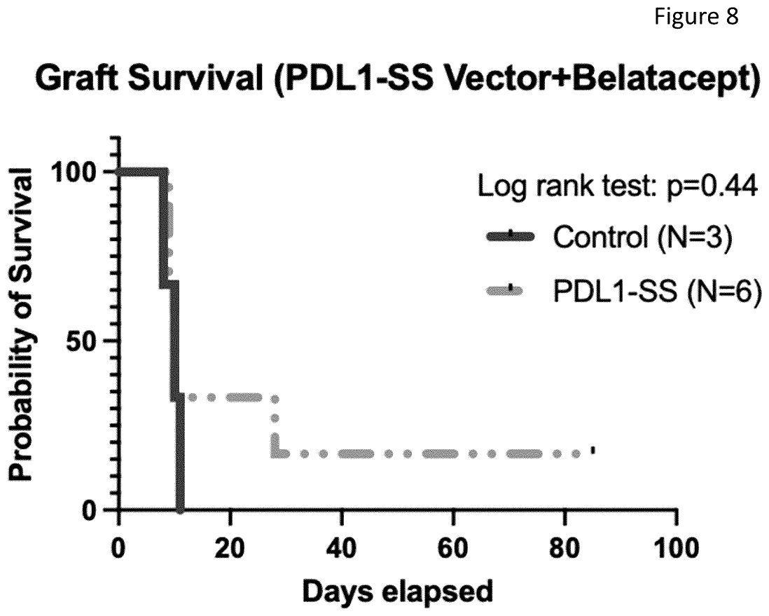

In our first set of experiments, we prioritized the use of PD-L1-TM transgenic hearts, due to their elevated level of PD-L1 surface expression. In these experiments, we transplanted hearts from C57BL6 into BALB/c recipients that we also treated with a single dose of Abatacept (250 ug/mouse the day of implantation). In this setting, PD-L1-TM expressing transgenic hearts were not rejected, in comparison to 50% of control hearts by 21 days post-transplant (FIG. 7). To test the potential of vector mediated delivery of this approach, PD-L1-SS expressing AAV vectors were generated and utilized to transduce mouse hearts in vivo (intravenous injection of 2x10E12vg of AAV per mouse, 14 days prior to transplantation). Hearts from these mice were then removed and utilized in major mismatch experiments along with a single dose of belatacept (less potent immunosuppression in mice) to elicit immune suppression (250 ug/mouse the day of implantation). These experiments revealed that PD-L1-SS transduced hearts survived significantly longer than control heart, which were all rejected by 14 days post-implantation (FIG. 8). Some of these hearts survived over 80 days before being sacrificed to assess cardiac tissue. These experiments demonstrate the potential for PD-L1-TM and PD-L1-SS to prevent solid organ rejection from genetically engineered transgenic organs, as well as through the use of viral vectors that can express these genes after tissue transduction ex vivo. While these experiments were performed in mice in cardiac tissue, we believe that this approach could be used for different organs in humans, as well as to potentially enable the use of xenotransplantation using the organs of different species.

### REFERENCES

1. Benjamin, E. J. et al. Heart Disease and Stroke Statistics-2017 Update: A Report From the American Heart Association. Circulation 135, e146-e603, (2017)
2. Bishawi et al. A normothermic ex vivo organ perfusion delivery method for cardiac transplantation gene therapy. Sci Rep. 2019 May 29; 9(1):8029. doi: 10.1038/s41598-019-43737-y.
3. Owonikoko, et al. Cardiac allograft rejection as a complication of PD-1 checkpoint blockade for cancer immunotherapy: a case report. Cancer Immunol Immunother. 2017 January; 66 (1): 45-50. doi: 10.1007/s00262-016-1918-2. Epub 2016 Oct. 22.
4. Lipson et al. Tumor Regression and Allograft Rejection after Administration of Anti-PD-1. N Engl J Med. 2016 Mar. 3; 374(9):896-8. doi: 10.1056/NEJMc1509268.
5. Bishawi et al. PD-1 and PD-L1 expression in cardiac transplantation. Cardiovasc Pathol. September-October 2021; 54:107331. doi: 10.1016/j.carpath.2021.107331. Epub 2021 Mar. 16.
6. Bracamonte-Baran, W, et al. Endothelial Stromal PD-L1 (Programmed Death Ligand 1) Modulates CD8+T-Cell Infiltration After Heart Transplantation. Circ Heart Fail. 2021 October; 14(10):e007982. doi: 10.1161/CIRCHEARTFAILURE.120.007982.
7. Buermann A, et al. Pigs expressing the human inhibitory ligand PD-L1 (CD 274) provide a new source of xenogeneic cells and tissues with low immunogenic properties. Xenotransplantation. 2018 September; 25(5):e12387. doi: 10.1111/xen.12387.
8 Ozkaynak et al. Programmed death-1 targeting can promote allograft survival. J Immunol. 2002 Dec. 1; 169(11):6546-53. doi: 10.4049/jimmunol.169.11.6546.
9. Yang et al. Critical role of donor tissue expression of programmed death ligand-1 in regulating cardiac allograft rejection and vasculopathy. Circulation. 2008 Feb. 5; 117(5):660-9. doi: 10.1161/CIRCULATIONAHA.107.741025. Epub 2008 Jan. 22.
10. Wang et al. Protective role of programmed death 1 ligand 1 (PD-L1) in nonobese diabetic mice: the paradox in transgenic models. Diabetes. 2008 July; 57(7):1861-9. doi: 10.2337/db07-1260. Epub 2008 Apr. 16.
11. Borges, T J, et al. Overexpression of PD-1 on T cells promotes tolerance in cardiac transplantation via ICOS-dependent mechanisms. JCI Insight. 2021 Dec. 22; 6(24):e142909. doi: 10.1172/jci.insight. 142909.
12. Paul P K, et al. Islet allografts expressing a PD-L1 and IDO fusion protein evade immune rejection and reverse preexisting diabetes in immunocompetent mice without systemic immunosuppression. Am J Transplant. 2022 Jul. 27. doi: 10.1111/ajt.17162.
13. Lucas et al. PD-L1 Reverse Signaling in Dermal Dendritic Cells Promotes Dendritic Cell Migration Required for Skin Immunity. Cell Rep. 2020 Oct. 13; 33(2):108258. doi: 10.1016/j.celrep.2020.108258.
14. Okuyama et al. Elevated Soluble PD-L1 in Pregnant Women's Serum Suppresses the Immune Reaction. Front Immunol. 2019 Feb. 18; 10:86. doi: 10.3389/fimmu.2019.00086. eCollection 2019.
15. Han et al. The clinical implication of soluble PD-L1 (sPD-L1) in patients with breast cancer and its biological function in regulating the function of T lymphocyte. Cancer Immunol Immunother. 2021 October; 70(10): 2893-2909. doi: 10.1007/s00262-021-02898-4. Epub 2021 Mar. 10.
16. Stewart S, et al. Revision of the 1990 working formulation for the standardization of nomenclature in the diagnosis of heart rejection. J Heart Lung Transplant 2005; 24(11):1710-20.

### Example 3

The present disclosure provides constructs and methods which may be used to transduce organs ex vivo with immunosuppressive genes. To illustrate this method of gene therapy, an article to be transplanted may be preserved by any method known in the art, including but not limited to a preservation method that maintains normothermic and aerobic metabolism of the article. The preserved article may be exposed ex vivo to one or more of the constructs described herein. These constructs may comprise a promoter operably linked to an immune checkpoint protein, or an extracellular portion of an immune checkpoint protein, such as PDL1, CTLA4, Galaectin-3, etc. These constructs may further comprise a secretory signal operably connected to the promoter and functionally linked to the nucleic acid encoding the immune checkpoint protein or extracellular portion of an immune checkpoint protein such that the immune checkpoint protein is secreted. For example, a heart may undergo normothermic ex vivo perfusion, wherein the perfusion may comprise an AAV vector. The AAV vector may encode a cell type promoter such as a cardiac promoter operably linked to a secretion signal and a nucleic acid encoding secreted PDL1. Expression of the vector or immunosuppressive gene may be evaluated following perfusion into the organ or article. The vector perfused organ can then be transplanted into a recipient. An organ that has been perfused ex vivo with a construct encoding an immunosuppressive gene is expected to improve transplantation outcomes, including decreased rejection of the transplanted organ, decreased immune response to the transplant and reduced ischemic injury. Ex vivo perfusion with a construct encoding an immunosuppressive gene may also decrease the amount, duration or dosing frequency of immunosuppressive therapy following transplantation. The methods and constructs described herein may also allow for the transplantation of organs with a greater degree of HLA mismatch. Without wishing to be bound by theory, an organ that is perfused ex vivo with a construct encoding an immunosuppressive gene, such as PDL1 or a variant thereof, may have a lower incidence of rejection and have a higher incidence of HLA mismatch than an organ not perfused with the construct. This approach may also have utility in suppressing immune mechanisms responsible for organ rejection of non-human organs, thus allowing more successful xenotransplantation through the modification of animal organs through genetic modification or through the use of ex vivo viral (or non-viral) transduction with vectors permitting the expression of these PD-L1 genes.

What is claimed:

1. A nucleic acid construct comprising a promotor operably connected to a nucleic acid sequence encoding an immune checkpoint protein or extracellular portion of an immune checkpoint protein, wherein the immune checkpoint protein or extracellular portion of an immune checkpoint protein is capable of binding to its receptor.

2. The construct of claim 1, further comprising a secretory signal operably connected to the promoter and functionally linked to the nucleic acid encoding the immune checkpoint protein or extracellular portion of an immune checkpoint protein.

3. The construct of claim 2, wherein the secretory signal encodes a signal peptide and allows for the extracellular portion of the immune checkpoint protein to be secreted.

4. The construct of any one of claims 1-3, wherein the immune checkpoint protein is selected from the group consisting of A2AR, A2BR, B7-H2, B7-H3, B7-H4, 2B4 (CD244), B7.1, B7.2, BTLA, CTLA4, ICOS, IDO, ITL-4, HVEM, KIR, LAG3, gp49B, NOX2, PD1, PDL1, PDL2, PIR-B, TIM-1, TIM-3, TIM-4, TIGIT, VISTA, SIGLEC7, CD47, CD48, CD39, CD73, CD160, CD200, HVEC, CEACAM1, CD155, LAG-3, HLA-E, and combinations thereof.

5. The construct of claim 1 or 2, wherein the immune checkpoint protein comprises PDL1.

6. The construct of claim 1 or 2, wherein the immune checkpoint protein comprises PD1.

7. The construct of claim 1, wherein the immune checkpoint protein comprises PDL1 or an extracellular portion thereof and has a sequence selected from the group consisting of SEQ ID NO: 8, SEQ ID NO: 10, SEQ ID NO: 12, a sequence with 95% identity to SEQ ID NO: 8, a sequence with 95% identity to SEQ ID NO: 10, a sequence with 95% identity to and SEQ ID NO: 12, and any fragment or variant thereof capable of binding to and activating PD1.

8. The construct of any one of the previous claims, wherein the immune checkpoint protein comprises CTLA4.

9. The construct of any one of the previous claims, wherein the promoter sequence encodes a constitutively active promoter, an inducible promoter, a tissue specific promoter, a cell-type specific promoter or a temporally restricted promoter.

10. The construct of any one of the previous claims, wherein the construct comprises a viral vector.

11. The construct of claim 10, wherein the viral vector is selected from the group consisting of retroviruses, adenoviruses, oncoretroviruses, lentiviruses, spumaviruses, adeno-associated viruses, herpes simplex viruses, and combinations thereof.

12. The construct of claim 11, wherein the viral vector comprises an adeno-associated (AAV) vector.

13. A pharmaceutical composition comprising the construct of any one of the preceding claims and a pharmaceutically acceptable carrier, diluent and/or excipient.

14. An ex vivo or in vitro method for expressing an immune checkpoint protein or portion thereof in a transplantable article, the method comprising introducing the construct of any one of claims 1-12 or the pharmaceutical composition of claim 13 into a transplantable article, optionally wherein introducing includes ex vivo or in vitro perfusion of the construct or the pharmaceutical composition into the article.

15. The method of claim 14, further comprising analyzing the article or cells of the article for the presence or expression of the construct or the immune checkpoint protein encoded by the construct in the article.

16. The method of claim 15, further comprising transplanting the article into a subject.

17. The method of claim 16, further comprising administering to the subject a therapeutically effective amount of one or more additional therapeutic agents.

18. The method of claim 17, wherein the one or more additional therapeutic agent is administered prior to, during or after the transplantation of the article to the subject.

19. The method of any one of claims 14-18, wherein the article is selected from the group consisting of an organ, a cell population, skin, and a tissue.

20. The method of claim 19, wherein the organ is selected from the group consisting of heart, lung, kidney, liver, pancreas, spleen, intestine, colon, eye, stomach, ovary, testes, bladder, uterus, adrenal glands, and combinations thereof.

21. An ex vivo method for preventing or reducing rejection of a transplanted article in a subject, the method comprising introducing the construct of any one of claims 1-12 or a pharmaceutical composition of claim 13 into a transplantable article prior to transplantation of the article into the subject.

22. The method of claim 21, further comprising analyzing the article or cells of the article for the presence or expression of the construct or the immune checkpoint protein encoded by the construct in the article.

23. The method of claim 22, further comprising transplanting the article into a subject.

24. The method of claim 23, wherein the one or more additional therapeutic agent is administered prior to, during or after the transplantation of the article to the subject.

25. The method of any one of claims 21-24, wherein the article for transplantation is selected from the group consisting of an organ, a cell population, skin, and a tissue.

26. The method of claim 25, wherein the organ is selected from the group consisting of heart, lung, kidney, liver, pancreas, spleen, intestine, colon, eye, stomach, ovary, testes, bladder, uterus, adrenal glands, and combinations thereof.

27. An ex vivo method for introducing a construct into a transplantable article, the method comprising ex vivo perfusion of the construct into the article prior to transplantation, wherein the construct comprises a promotor operably connected to a nucleic acid sequence encoding an immune checkpoint protein or extracellular portion of an immune checkpoint protein, wherein the immune checkpoint protein or extracellular portion of an immune checkpoint protein is capable of binding to its receptor.

28. The method of claim 27, further comprising transplanting the article into a subject.

29. The method of claim 27 or 28, wherein the article is selected from the group consisting of an organ, a cell population, skin, and a tissue.

30. The method of claim 29, wherein the organ is selected from the group consisting of heart, lung, kidney, liver, pancreas, spleen, intestine, colon, eye, stomach, ovary, testes, bladder, uterus, adrenal glands, and combinations thereof.

31. The method of claim 27, wherein the construct further comprises a secretory signal operable connected to the promoter and functionally linked to the nucleic acid encoding the immune checkpoint protein or extracellular portion of an immune checkpoint protein, and wherein the secretory signal encodes a signal peptide and allows for the immune checkpoint protein or extracellular portion of an immune checkpoint protein to be secreted.

32. The method of claim 27 or 31, wherein the immune checkpoint protein is selected from the group consisting of A2AR, A2BR, B7-H2, B7-H3, B7-H4, 2B4 (CD244), B7.1, B7.2, BTLA, CTLA4, ICOS, IDO, ITL-4, HVEM, KIR, LAG3, gp49B, NOX2, PD1, PDL1, PDL2, PIR-B, TIM-1, TIM-3, TIM-4, TIGIT, VISTA, SIGLEC7, CD47, CD48, CD39, CD73, CD160, CD200, HVEC, CEACAM1, CD155, LAG-3, HLA-E, and combinations thereof.

33. The method of claim 32, wherein the immune checkpoint protein comprises PDL1 or an extracellular portion thereof and has a sequence selected from the group consisting of SEQ ID NO: 8, SEQ ID NO: 10, SEQ ID NO: 12, a sequence with 95% identity to SEQ ID NO: 8, a sequence with 95% identity to SEQ ID NO: 10, a sequence with 95% identity to and SEQ ID NO: 12, and any fragment or variant thereof capable of binding to and activating PD1.

34. The method of claim 27, wherein the construct is a viral vector, and the viral vector is selected from the group consisting of retroviruses, adenoviruses, oncoretroviruses, lentiviruses, spumaviruses, adeno-associated viruses, herpes simplex viruses, and combinations thereof.

35. An ex vivo method for preventing or reducing rejection of a transplanted article in a subject, the method comprising introducing a construct into a transplantable article prior to transplantation into a subject, wherein the construct encodes a promotor operably connected to a nucleic acid sequence encoding an immune checkpoint protein or extracellular portion of an immune checkpoint protein, wherein the immune checkpoint protein or extracellular portion of an immune checkpoint protein is capable of binding to its receptor.

36. The method of claim 35, further comprising transplanting the article into a subject.

37. The method of claim 35 or 36, wherein the article is selected from the group consisting of an organ, a cell population, skin, and a tissue.

38. The method of claim 37, wherein the organ is selected from the group consisting of heart, lung, kidney, liver, pancreas, spleen, intestine, colon, eye, stomach, ovary, testes, bladder, uterus, adrenal glands, and combinations thereof.

39. The method of claim 35, wherein the construct further comprises a secretory signal operable connected to the promoter and functionally linked to the nucleic acid encoding the immune checkpoint protein or extracellular portion of an immune checkpoint protein, and wherein the secretory signal encodes a signal peptide and allows for the immune checkpoint protein or extracellular portion of an immune checkpoint protein to be secreted.

40. The method of any one of claims 35-39, wherein the immune checkpoint protein is selected from the group consisting of A2AR, A2BR, B7-H2, B7-H3, B7-H4, 2B4 (CD244), B7.1, B7.2, BTLA, CTLA4, ICOS, IDO, ITL-4, HVEM, KIR, LAG3, gp49B, NOX2, PD1, PDL1, PDL2, PIR-B, TIM-1, TIM-3, TIM-4, TIGIT, VISTA, SIGLEC7, CD47, CD48, CD39, CD73, CD160, CD200, HVEC, CEACAM1, CD155, LAG-3, HLA-E, and combinations thereof.

41. The method of claim 40, wherein the immune checkpoint protein comprises PDL1 or an extracellular portion thereof and has a sequence selected from the group consisting of SEQ ID NO: 8, SEQ ID NO: 10, SEQ ID NO: 12, a sequence with 95% identity to SEQ ID NO: 8, a sequence with 95% identity to SEQ ID NO: 10, a sequence with 95% identity to and SEQ ID NO: 12, and any fragment or variant thereof capable of binding to and activating PD1.

42. The method of claim 41, wherein the construct is a viral vector, and the viral vector is selected from the group consisting of retroviruses, adenoviruses, oncoretroviruses, lentiviruses, spumaviruses, adeno-associated viruses, herpes simplex viruses, and combinations thereof.


SEQUENCE LISTING (13 sequences)

SEQ ID NO: 1 - DNA, 873 bp, synthetic construct
  source 1..873: mol_type = other DNA
atgaggatatttgctggcattatattcacagcctgctgtcacttgctacgggcgtttactatcacggctccaaaggacttgtacgtggtggagtatggcagcaacgtcacgatggagtgcagattccctgtagaacgggagctggacctgcttgcgttagtggtgtactgggaaaaggaagatgagcaagtgattcagtttgtggcaggagaggaggaccttaagcctcagcacagcaacttcagggggagagcctcgctgccaaaggaccagcttttgaagggaaatgctgcccttcagatcacagacgtcaagctgcaggacgcaggcgtttactgctgcataatcagctacggtggtgcggactacaagcgaatcacgctgaaagtcaatgccccataccgcaaaatcaaccagagaatttccgtggatccagccacttctgagcatgaactaatatgtcaggccgagggttatccagaagctgaggtaatctggacaaacagtgaccaccaacccgtgagtgggaagagaagtgtcaccacttcccggacagaggggatgcttctcaatgtgaccagcagtctgagggtcaacgccacagcgaatgatgttttctactgtacgttttggagatcacagccagggcaaaaccacacagcggagctgatcatcccagaactgcctgcaacacatcctccacagaacaggacccactgggtgcttctgggatccatcctgttgttcctcattgtagtgtccacggtcctcctcttcttgagaaaacaagtgagaatgctagatgtggagaaatgtggcgttgaagatacaagctcaaaaaaccgaaatgatacacaattcgaggagacgtaa

SEQ ID NO: 2 - protein, 290 aa, synthetic construct
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 3 - DNA, 783 bp, synthetic construct
  source 1..783: mol_type = other DNA
atgaggatatttgctggcattatattcacagcctgctgtcacttgctacgggcgtttactatcacggctccaaaggacttgtacgtggtggagtatggcagcaacgtcacgatggagtgcagattccctgtagaacgggagctggacctgcttgcgttagtggtgtactgggaaaaggaagatgagcaagtgattcagtttgtggcaggagaggaggaccttaagcctcagcacagcaacttcagggggagagcctcgctgccaaaggaccagcttttgaagggaaatgctgcccttcagatcacagacgtcaagctgcaggacgcaggcgtttactgctgcataatcagctacggtggtgcggactacaagcgaatcacgctgaaagtcaatgccccataccgcaaaatcaaccagagaatttccgtggatccagccacttctgagcatgaactaatatgtcaggccgagggttatccagaagctgaggtaatctggacaaacagtgaccaccaacccgtgagtgggaagagaagtgtcaccacttcccggacagaggggatgcttctcaatgtgaccagcagtctgagggtcaacgccacagcgaatgatgttttctactgtacgttttggagatcacagccagggcaaaaccacacagcggagctgatcatcccagaactgcctgcaacacatcctccacagaacaggacccactgggtgcttctgggatccatcctgttgttcctcattgtagtgtccacggtcctcctcttcttgtaa

SEQ ID NO: 4 - protein, 260 aa, synthetic construct
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFL

SEQ ID NO: 5 - DNA, 723 bp, synthetic construct
  source 1..723: mol_type = other DNA
atgatgaagttgatccagctttgcatcttattctggtgctggagagcaatctgctgctttactatcacggctccaaaggacttgtacgtggtggagtatggcagcaacgtcacgatggagtgcagattccctgtagaacgggagctggacctgcttgcgttagtggtgtactgggaaaaggaagatgagcaagtgattcagtttgtggcaggagaggaggaccttaagcctcagcacagcaacttcagggggagagcctcgctgccaaaggaccagcttttgaagggaaatgctgcccttcagatcacagacgtcaagctgcaggacgcaggcgtttactgctgcataatcagctacggtggtgcggactacaagcgaatcacgctgaaagtcaatgccccataccgcaaaatcaaccagagaatttccgtggatccagccacttctgagcatgaactaatatgtcaggccgagggttatccagaagctgaggtaatctggacaaacagtgaccaccaacccgtgagtgggaagagaagtgtcaccacttcccggacagaggggatgcttctcaatgtgaccagcagtctgagggtcaacgccacagcgaatgatgttttctactgtacgttttggagatcacagccagggcaaaaccacacagcggagctgatcatcccagaactgcctgcaacacatcctccacagaacaggacccactaa

SEQ ID NO: 6 - protein, 240 aa, synthetic construct
MMKLIQLCILFWCWRAICCFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTH

SEQ ID NO: 7 - DNA, 873 bp, synthetic construct
  source 1..873: mol_type = other DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 8 - protein, 290 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 9 - DNA, 780 bp, synthetic construct
  source 1..780: mol_type = other DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttctaa

SEQ ID NO: 10 - protein, 259 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIF

SEQ ID NO: 11 - DNA, 738 bp, synthetic construct
  source 1..738: mol_type = other DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccaggtaatattctgaatgtgtccattaaaatatgtctaacactgtcccctagcacctag

SEQ ID NO: 12 - protein, 245 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 13 - DNA, 57 bp, synthetic construct
  source 1..57: mol_type = other DNA
atgatgaagttgatccagctttgcatcttattctggtgctggagagcaatctgctgc